(19) United States
(12) Patent Application Publication
(10) Pub. No.: US 2015/0112377 A1
(43) Pub. Date: Apr. 23, 2015

(54) EMBOLIC PROTECTION SYSTEM

(71) Applicants: Joshua Clay Arnone, Columbia, MO (US); Kyle D. Rood, Saint Peters, MO (US); Clint Mathews, Columbia, MO (US); Raja R. Gopaldas, Columbia, MO (US); Charles Sweat, JR., Hampton, VA (US); Jain Khushbu, Santa Clara, CA (US)

(72) Inventors: Joshua Clay Arnone, Columbia, MO (US); Kyle D. Rood, Saint Peters, MO (US); Clint Mathews, Columbia, MO (US); Raja R. Gopaldas, Columbia, MO (US); Charles Sweat, JR., Hampton, VA (US); Jain Khushbu, Santa Clara, CA (US)

(73) Assignee: THE CURATORS OF THE UNIVERSITY OF MISSOURI, Columbia, MO (US)

(21) Appl. No.: 14/399,761

(22) PCT Filed: May 3, 2013
(86) PCT No.: PCT/US2013/039523

§ 371 Date: Nov. 7, 2014

Related U.S. Application Data
(60) Provisional application No. 61/688,110, filed on May 8, 2012.

(51) Int. Cl. A61F 2/01

(52) U.S. Cl. CPC A61F 2/01; A61F 2002/011
USPC 606/200

(57) ABSTRACT

A collapsible blood filtering aortic arch bridge comprising a dumbbell shaped chassis having a tubular waist, a first conical end, and a second conical end such that only a periphery of the first and second ends contact the intima of an aortic arch when the bridge is disposed and expanded within the aortic arch of a patient. The waist is flexible so that the bridge can bend to comply with the curvature of the aortic arch. The bridge additionally comprises a blood filtering sleeve disposed over an interior or an exterior of the chassis for filtering blood flowing through the bridge into aortic arch vessels of the patient when the bridge is disposed within the aortic arch. Furthermore, the bridge comprises a retrieval sleeve disposed over the exterior of the chassis for collapsing the bridge to a cylindrical form for retrieval of the bridge from the aortic arch.

### CROSS-REFERENCE TO RELATED APPLICATIONS

This application is a National Stage of International Application No. PCT/US2013/039523, filed May 3, 2013, which claims priority to U.S. Provisional Application No. 61/688,110, filed on May 8, 2012. The disclosures of the above applications are incorporated herein by reference in their entirety.

### FIELD

The present teachings relate to a system for protecting aortic arch vessels during cardiac procedures, endovascular cardiac and aortic interventions, and non-operative treatment of infective endocarditis.

### BACKGROUND

The statements in this section merely provide background information related to the present disclosure and may not constitute prior art.

The current rate of cerebrovascular stroke during aortic valve replacement procedures using open, minimally invasive, or endovascular approaches is known to be as high as 22%. Currently there are no FDA approved devices for use in the United States designed to prevent cerebrovascular stroke during heart valve replacement, and only two devices are available in Europe. However, the known devices have serious deficiencies. For example, they are unreliable for creating a seal over the main vessel junctions within the aortic intima. This creates the opportunity for embolic particles to travel through the area of the compromised seal into the aortic arch arteries potentially causing a cerebrovascular stroke. Additionally, such known devices typically fail to provide a smooth transition between the devices and the intimal interface, which can result in stagnant blood flow at the interface and increase the risk for formation of stroke-causing emboli. Furthermore, such known devices do not trap embolic vegetations associated with endocarditis, and hence, do not decrease the risk of neurological dysfunction.

### SUMMARY

The present disclosure provides an embolic protection system for aortic arch vessels during a cardiac procedure and non-operative treatment of endocarditis.

In various embodiments, the present disclosure provides a collapsible blood filtering aortic arch bridge comprising a dumbbell shaped chassis structured to provide the bridge with a tubular waist, a first conical end formed and a second conical end such that only a periphery of the first and second ends contact the intima of an aortic arch when the bridge is disposed and expanded within the aortic arch of a patient. The chassis is structured and operable to bend to comply with the curvature of the aortic arch of the patient. The bridge additionally comprising a blood filtering sleeve disposed over an interior or an exterior of the chassis and structured and operable to filter blood flowing through the bridge into aortic arch vessels of the patient when the bridge is disposed within the aortic arch. Furthermore the bridge comprises a retrieval sleeve disposed over the exterior of the chassis. The retrieval sleeve is structured and operable to collapse the bridge to a cylindrical form for retrieval of the bridge from the aortic arch.

In various other embodiments, the present disclosure provides an embolic protection system comprising a collapsible blood filtering aortic arch bridge and a bridge retrieval tool. The blood filtering bridge is structured and operable to bend to comply with the curvature of an aortic arch of a patient into which the bridge is disposable. The bridge comprises a chassis that is expandable and collapsible, wherein the chassis is structured to provide the bridge with a dumbbell-like shape when expanded, whereby the chassis has a tubular waist, a first conical end formed at a first end of the waist, and a second conical end formed at an opposing second end of the waist such that only a periphery of the first and second ends contact the intima of the aortic arch when the bridge is disposed and expanded within the aortic arch. The bridge additionally comprises a blood filtering sleeve attached to the chassis. The blood filtering sleeve is structured and operable to filter blood flowing through the bridge into aortic arch vessels of the patient when the bridge is disposed and expanded within the aortic arch. Furthermore, the bridge comprises a retrieval sleeve disposed over an exterior of the chassis that is structured and operable to collapse the bridge to a cylindrical form for retrieval of the bridge from the aortic arch.

The bridge retrieval tool is structured and operable to retrieve the bridge from disposition within the aortic arch. In various implementations, the retrieval tool comprises a multi-layer catheter including a retention wire concentrically disposed within a movable outer sheath and a bridge connector connected to a distal end of the retention wire. The bridge connector is structured and operable to connect with the retrieval sleeve to retrieve the bridge from disposition within the aortic arch. The tool additionally comprises a control handle connected to the catheter that is structured and operable to control longitudinal movement of both the retention wire and the outer sheath.

Further areas of applicability of the present teachings will become apparent from the description provided herein. It should be understood that the description and specific examples are intended for purposes of illustration only and are not intended to limit the scope of the present teachings.

### DRAWINGS

The drawings described herein are for illustration purposes only and are not intended to limit the scope of the present teachings in any way.

FIG. 1 is schematic of an embolic protection system, in accordance with various embodiments of the present disclosure.

FIG. 2 is a schematic of a heart having a collapsible blood filtering aortic arch bridge of the of embolic protection system shown in FIG. 1 disposed within the aortic arch, in accordance with various embodiments of the present disclosure.

FIG. 3 is an isometric view of the collapsible blood filtering aortic arch bridge of the embolic protection system shown in FIGS. 1 and 2, the bridge shown in an expanded state, in accordance with various embodiments of the present disclosure.

FIG. 4 is an isometric view of the collapsible blood filtering aortic arch bridge shown in FIG. 3, the bridge shown in a collapsed state, in accordance with various embodiments of the present disclosure.

FIG. 5A is side view of a shape memory material chassis of the collapsible blood filtering aortic arch bridge shown in FIGS. 3 and 4, the chassis shown in the expanded and collapsed states, in accordance with various embodiments of the present disclosure.

FIG. 5B is a side view of the shape memory material chassis shown in FIG. 5A having a blood filtering sleeve disposed over an interior of the chassis, the chassis with the blood filtering sleeve shown in the expanded and collapsed states, in accordance with various embodiments of the present disclosure.

FIG. 5C is side view of the shape memory material chassis and blood filtering sleeve shown in FIG. 5B having a retrieval sleeve disposed over an exterior of the chassis providing the aortic arch bridge shown in FIGS. 3 and 4, the aortic arch bridge shown in the expanded and collapsed states, in accordance with various embodiments of the present disclosure.

FIG. 6 is a cross-sectional view of the blood filtering aortic arch bridge shown in FIG. 3, in accordance with various embodiments of the present disclosure.

FIG. 7 is an isometric view of a bridge retrieval tool of the embolic protection system shown in FIG. 1, in accordance with various embodiments of the present disclosure.

FIG. 8 is a side view of a bridge coupling mechanism of the bridge retrieval tool shown in FIG. 7, in accordance with various embodiments of the present disclosure.

FIG. 9 is a cross-sectional view of the bridge coupling mechanism shown in FIG. 8, in accordance with various embodiments of the present disclosure.

FIG. 10 is a cross-sectional view of the bridge coupling mechanism shown in FIGS. 8 and 9 having a magnetic button of the blood filtering aortic arch bridge, shown in FIG. 3, magnetically coupled to a magnetic bridge connector of the bridge coupling mechanism, in accordance with various embodiments of the present disclosure.

FIG. 11 is a cross-sectional view of the bridge coupling mechanism having the magnetic button magnetically coupled with the magnetic bridge connector, as shown in FIG. 10, and pulled into a locking claw of the bridge coupling mechanism, in accordance with various embodiments of the present disclosure.

FIG. 12 is a cross-sectional view of the magnetic button magnetically coupled with the magnetic bridge connector and pulled into the locking claw of the bridge coupling mechanism, as shown in FIG. 11, having an outer sleeve of a multi-layer catheter of the bridge retrieval tool extended over the bridge coupling mechanism, in accordance with various embodiments of the present disclosure.

FIG. 13 is a cross-sectional view of a portion of a control handle of the bridge retrieval tool shown in FIG. 7, in accordance with various embodiments of the present disclosure.

FIG. 14A is a side-view of the control handle shown in FIG. 13 having a thumb control pad of the control handle in a bridge connection position, in accordance with various embodiments of the present description.

FIG. 14B is a side-view of the control handle shown in FIG. 13 having the thumb control pad in a bridge securing position, in accordance with various embodiments of the present description.

FIG. 14C is a side-view of the control handle shown in FIG. 13 having the thumb control pad in a bridge collapsing position, in accordance with various embodiments of the present description.

FIG. 15 is a schematic of an aorta having the collapsible blood filtering aortic arch bridge, shown in FIG. 3, disposed and in an expanded state therein, in accordance with various embodiments of the present disclosure.

FIG. 16 is a schematic illustrating the collapsible blood filtering aortic arch bridge disposed within the aorta and being progressively collapsed as an outer sheath of the multi-layer catheter of the bridge retrieval tool is advanced over the collapsing bridge, in accordance with various embodiments of the present disclosure.

FIG. 17 is a schematic illustrating the outer sheath of the multi-layer catheter advanced over the entire collapsed blood filtering aortic arch bridge, in accordance with various embodiments of the present disclosure.

FIG. 18 is an isometric view of a bridge retrieval tool shown in FIG. 7, including an expandable outer sheath tip, in accordance with various embodiments of the present disclosure.

Corresponding reference numerals indicate corresponding parts throughout the several views of drawings.

### DETAILED DESCRIPTION

The following description is merely exemplary in nature and is in no way intended to limit the present teachings, application, or uses. Throughout this specification, like reference numerals will be used to refer to like elements.

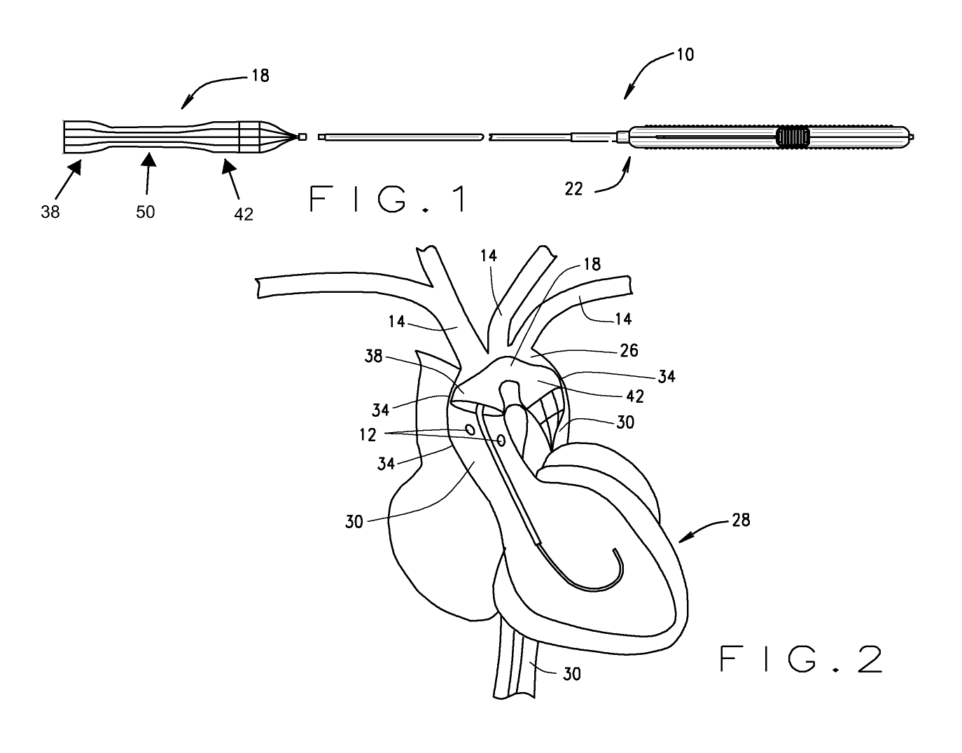

Referring to FIGS. 1 and 2, the present disclosure provides an embolic protection system 10 that is structured and operable to prevent embolic particles (emboli) 12 generated during aortic valve replacement from traveling into the aortic arch arteries 14 potentially causing a cerebrovascular stroke. Generally, the system 10 includes a collapsible blood filtering aortic arch bridge 18 and a bridge retrieval tool 22. The bridge 18 is structured and operable to be disposed within the aortic arch 26 and span the juncture of the aortic arch arteries 14 with the aortic arch 26 such that substantially all the blood flowing from the heart 28 through the aorta 30 and into the aortic arch arteries 14 will pass through, and be filtered by, the bridge 18, as described further below. Importantly, the bridge 18 is further structured and operable to be very flexible such that the bridge will bend or contour to comply with the anatomy, e.g., curvature, of the aortic arch 26 and not distort the anatomy of the aortic arch 26, all the while being structured and operable to establish and maintain a tight seal between the aortic intima 34 and the ends 38 and 42 of the bridge 18 such that emboli 12 cannot pass between the intima 34 and ends 38 and 42, but will be forced to travel through the bridge 18 and thereby prevented from traveling into the arch arteries 14. Also, importantly, the bridge 18 is formed or structured to have a substantially dumbbell-like shape having a tubular waist 50 with a first, or upstream, conical end 38 formed at one end of the waist 50 and a second, or downstream, conical end 42 formed at the opposing end of the waist such that only a periphery of the upstream and downstream ends 38 and 42 contact the intima 34 when the bridge 18 is disposed and expanded within the aortic arch 26.

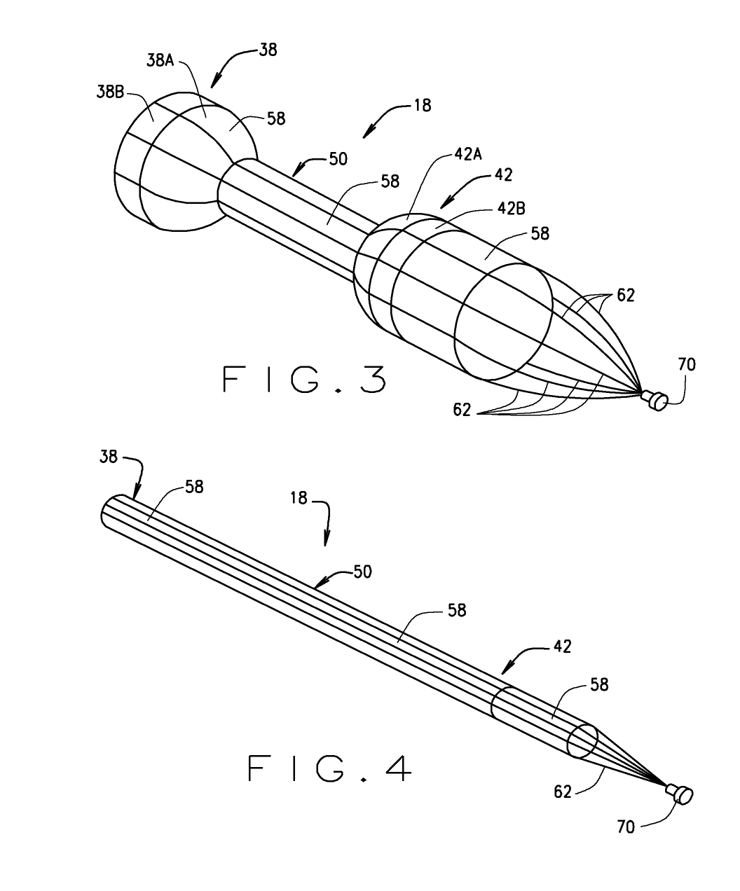

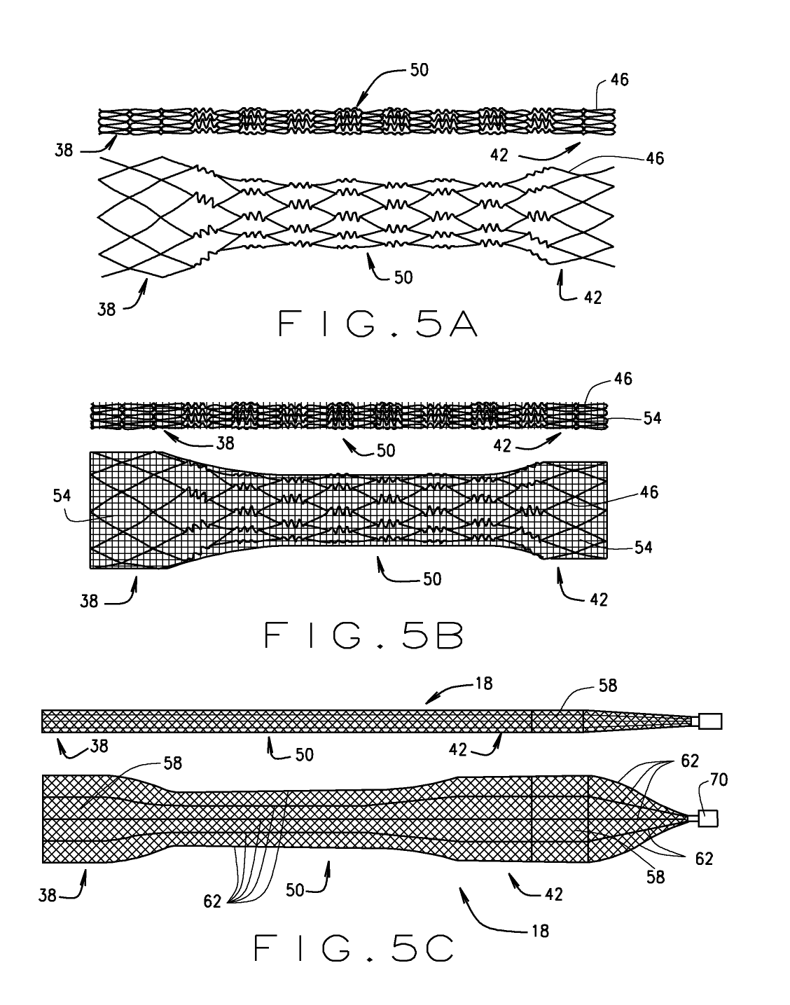

Referring now to FIGS. 3 through 6, as described in detail below, the bridge 18 is structured and operable to have the dumbbell-like shape when in an expanded state, as illustrated in FIG. 3, and is collapsible to a hollow cylindrical form when in a collapsed state, as illustrated in FIG. 4. More specifically, the bridge 18 comprises a chassis 46 (FIG. 5A) that is naturally biased to the expanded state but easily transformable to the collapsed state, an elastic blood filtering sleeve 54 that covers and is attached to either an interior or exterior of the chassis 46 (FIG. 5B), and an elastic retrieval sleeve 58 disposed over the exterior of the chassis 46 (FIG. 5C). In the embodiments wherein the blood filtering sleeve 54 is disposed over the exterior of the chassis 46, the retrieval sleeve 58 is disposed over the exterior of the chassis 46 and the blood filtering sleeve 54.

As described above, the chassis 46 is structured or formed to have the dumbbell-like shape when in the expanded state. Therefore, it should be understood that the chassis 46 provides and defines the waist 50, the upstream conical end 38 and the downstream conical end 42 of the bridge 18 when the bridge 18 is in the expanded state, and provides and defines the hollow cylindrical shape of the bridge 18 when the bridge 18 is in the collapsed state.

In various embodiments, the chassis 46 is fabricated of a shape memory material, e.g., nitinol, to have the dumbbell-like shape, but is collapsible to the cylindrical shape, as illustrated in FIG. 5A. Hence, the chassis 46 will naturally, i.e., without any external or environmental influences, assume the dumbbell-like shape, but can be easily compressed to have the hollow cylindrical shape. For example, in various embodiments, the chassis 46 can be fabricated by laser-cutting and shape-setting a small tube of shape memory alloy, e.g., nitinol, that forms a dumbbell shaped cage that can be easily compressed from the expanded state to the collapsed state, as exemplarily illustrated in FIG. 5A. Particularly, in various implementations, the chassis 46 can be fabricated by laser-cutting small (e.g., 4 mm) shape memory alloy (e.g., nitinol) tube and shape-setting the tube to acquire the expanded dumbbell shape. Radial sinusoidal rings can be formed at the conical ends 38 and 42, and periodically within the smaller waist 50, (i.e., the mid-section) to provide radial stability. Additionally, longitudinal sinusoidal struts can be formed along the length of the chassis 46 to provide longitudinal stability/flexibility. Furthermore, the strut pattern of the chassis 46 is structured and operable to prevent the outer sheath 98 of the retrieval tool catheter 74 (described below with regard to FIGS. 7-17) from binding with, snagging on, or catching on the bridge 18 during retrieval, as described below.

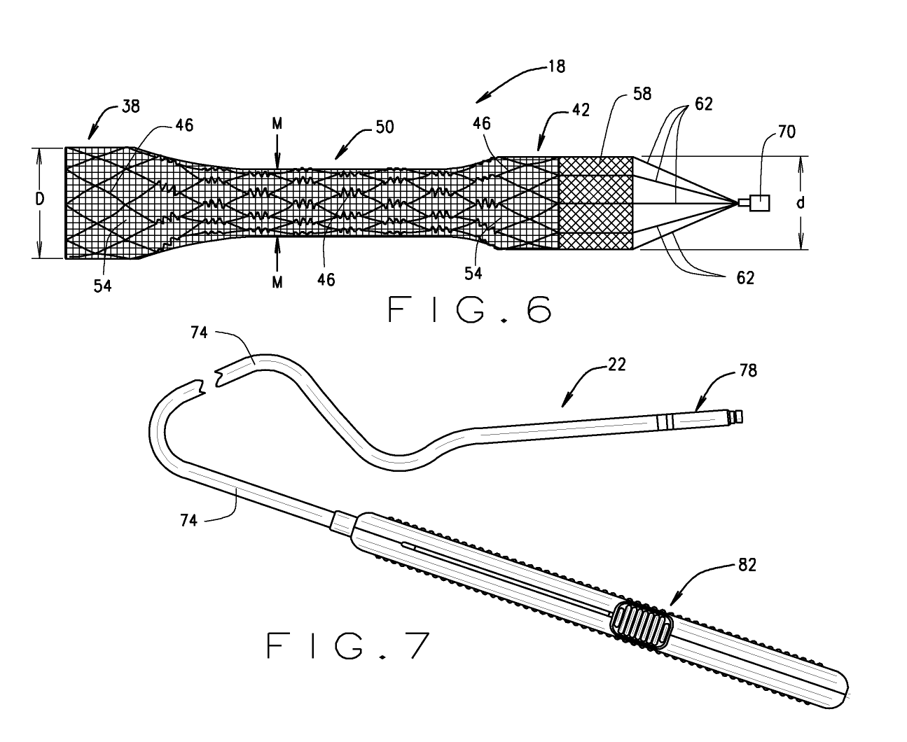

Additionally, in various embodiments, the chassis 46 is structured or formed such that the conical upstream end 38 of the bridge 18 has an outside diameter D that is greater than an outside diameter d of the conical downstream end 42 such that the bridge 18 conforms or accommodates the anatomy of the aorta arch 26 (illustrated in FIG. 6). That is, the difference in outside diameters D and d of the upstream and downstream ends 38 and 42 of the bridge 18 are structure and operable to accommodate the change of the inside diameter of the aorta 30 upstream of the aortic arch arteries (i.e., the portion of the aorta 30 extending between the heart 28 and the arch arteries 14) and the inside diameter of the aorta 30 downstream of the aortic arch arteries 14 (i.e., the portion of the aorta 30 extending between the arch arteries 14 and the abdomen). Thus, the upstream and downstream ends 38 and 42 are structured or formed to have outside diameters D and d, respectively, designed to fit the range of human aorta diameters without rupturing smaller aorta yet keeping the bridge 18 in place within larger aorta. For example, in various embodiments, the outside diameter D of the upstream end 38 is 20% to 40% larger than the outside diameter d of the downstream end 42.

Furthermore, the chassis 46 is structured or formed to provide and define the waist 50 of the bridge 18 to have an outside diameter M (shown in FIG. 6) that is smaller than the end outside diameters D and d. Therefore, importantly, when the bridge 18 is dispose within the aortic arch 26, wherein the bridge 18 will bend to accommodate the contour of the aortic arch 26, the exterior of the waist 50 will not come into contact with the aortic intima 34, thereby preventing the development of micro clots along the length of the waist 50. More particularly, the chassis 46 is structured or formed such that when the bridge 18 is disposed within the aortic arch 26, the waist 50 will generally be positioned or disposed in the mid-lumen or generally centered within the aorta 30. Therefore, blood will flow through the bridge 18 and pass through the sides of the waist 50 and the conical portion of the upstream end 38 into the arch arteries 14 without the occurrence of blood stagnation between the intima 34 and the bridge 18, thereby preventing the formation of micro-clots that could hazardously enter the arch arteries 14. Still further, the chassis 46 is structured or formed to provide the waist 50 outside diameter M that is large enough to provide an inside diameter of the waist 50 that is large enough to allow catheters and artificial aortic valves to pass through the bridge 18. Furthermore, the reduced diameter waist acts as a barrier between instrumentation that subsequently passes through the bridge 18 during medical/surgical procedures, e.g., aortic valve replacement procedures, and the intima, reducing the likelihood that the bridge 18 or instrumentation will irritate or damage the intima 34.

Additionally, as illustrated in FIG. 3, the conical upstream end 38 of the bridge 18 is structured to have a funnel portion 38A and a substantially cylindrical end rim portion 38B extending from the funnel portion 38A. Similarly, the conical downstream end 42 of the bridge 18 is structured to have a funnel portion 42A and a substantially cylindrical end rim portion 42B extending from the funnel portion 42A. Importantly, the exterior portion of the end rim portions 38B and 42B are the only part of the bridge 18 that contact the aortic intima 34 when the bridge 18 is disposed in the aortic arch 26.

Referring now to FIGS. 5B and 6, as described above, the bridge 28 comprises the elastic blood filtering sleeve 54 that is attached to and covers either the interior or the exterior of the chassis 46. Although the blood filtering sleeve 54 can be disposed over and attached to the exterior of the chassis 46 without departing from the scope of the present disclosure, for clarity and simplicity, the bridge 18 will be described herein as having the filtering sleeve 54 disposed within and attached to the interior of the chassis 46.

The blood filtering sleeve 54 is fabricated of a biocompatible material, e.g., polyethylene terephthalate (PET), and is fabricated to allow blood flowing into and through the bridge 18 to pass through the blood filtering sleeve 54, i.e., through the pores or openings of the sleeve 54, along the entire length of the bridge 18 between the upstream and downstream conical end rim portions 38B and 42B. Importantly, blood filtering sleeve 54 is fabricated such that the blood will flow through the blood filtering sleeve 54 into the aortic arch vessels 14 without a reduction in blood pressure nor a reduction in flow volume, while filtering the blood to prevent any emboli 12 from flowing into the arch vessels 14.

For example, in various embodiments, the blood filtering sleeve 54 is fabricated of a knitted (as opposed to woven or braided) biocompatible material, e.g., PET, to provide a knitted mesh. Utilizing a knitted material provides that the blood filtering sleeve 54 can expand and contract generally only in the longitudinal direction (i.e., along a longitudinal axis of the bridge 18). Hence, expansion of the bridge 18 from the collapsed state to the expanded state will not increase the porosity of the blood filtering sleeve 54. That is, the knitted mesh blood filtering sleeve 54 will easily stretch only in the longitudinal direction such that when the bridge 18 is expanded to the expanded state, the size and area of the openings or pores of the knitted mesh will not change. Rather, the openings or pores in the mesh will only elongate in a longitudinal direction, i.e., along a longitudinal axis of the bridge 18, but the size of the openings or pores lateral direction, i.e., orthogonal to the longitudinal axis of the bridge 18, will not change. Therefore, the blood filtering sleeve 54 will maintain its filtering capabilities and not allow larger size emboli 12 to flow through the blood filtering sleeve 54. In various embodiments, the blood filtering sleeve 54 is fabricated of knitted PET to have a porosity of between 50 to 300 microns, e.g., 100 micron, when the bridge 18 is deployed and in the expanded state. Additionally, utilizing a knitted material allows the blood filtering sleeve 54 to stretch as the chassis 46 bends during disposition within the aortic bridge 26. Furthermore, in various implementations, the blood filter sleeve 54 will be disposed within and attached to the chassis 46 such that the blood filter sleeve 54 will be a relaxed state when the chassis 46/bridge 18 is in the expanded state and will be “bunched” within the chassis 46 with the chassis 46/bridge 18 is in the collapsed state.

Additionally, the conical structure of the upstream end 38 is designed such that the luminal blood flow, i.e., the blood flowing from the heart, into upstream end 38, will contact the blood filtering sleeve 54 covering either the interior or exterior of the funnel portion 38A. Moreover, the contact of the blood flow with the blood filtering sleeve 54 covering the funnel portion 38A of the chassis 46 will apply a longitudinal force (i.e., a force parallel to the direction of the blood flow) to the funnel portion 38A. Subsequently, due to the conical structure of the funnel portion 38A, the longitudinal force of the blood flow against blood filtering sleeve 54 will result in lateral, or radially outward, forces exerted on the funnel portion 38A, which will in turn exert a lateral, or radially outward, force on the cylindrical end rim portion 38B. Importantly, this lateral, or radially outward, force exerted on the end rim portion 38 by the blood flowing into the bridge 18 will push, or press, the end rim portion 38 firmly against the aortic intima 34 such that the bridge 18 will be securely retained, or anchored, within the aortic arch 26, thereby preventing migration of the bridge 18 within the aortic arch 26, until the bridge 18 is removed using the retrieval tool 22, as described below. Furthermore, the conical ends 38 and 42 function to decrease the parallelism between the direction of the luminal blood flow and the walls of the bridge 18. Therefore, the blood can flow through the blood filtering sleeve 54 anywhere between the upstream and downstream conical end rim portions 38B and 42B, thereby significantly minimizing emboli causing stagnation along the interior and/or exterior surface of the bridge 18.

Referring now to FIGS. 5C and 6, as described above, the bridge 18 comprises the retrieval sleeve 58 disposed over the exterior of the chassis 46, or over the blood filtering sleeve 54 in the embodiments wherein the blood filtering sleeve 54 is disposed over the exterior of the chassis 46. The retrieval sleeve 58 is attached (e.g. sutured) to the upstream end 38 of chassis 46 such that the chassis 46 and blood filtering sleeve 54 are free to move or slide within the remainder of the retrieval sleeve 58. The retrieval sleeve 58 is structured and operable to be connectable to the bridge retrieval tool 22 and to collapse the bridge 18 from the expanded state to the collapsed state such that the bridge 18 can be retrieved, or removed, when desired.

The retrieval sleeve 58 is fabricated of a biocompatible material and is fabricated to allow blood flowing through the blood filtering sleeve 54, as described above, to flow into the aortic arch vessels 14 without a reduction in blood pressure nor a reduction in flow volume. For example, in various embodiments, the retrieval sleeve 58 is fabricated to have a porosity of between 200 to 500 microns when the bridge 18 is deployed and in the expanded state. The retrieval sleeve 58 is fabricated of a braided (as opposed to knitted or woven) biocompatible material to provide a braided mesh. For example, in various implementations, the retrieval sleeve 58 comprises a braided strong polymer monofilament such as polypropylene. Utilizing a braided material provides that a longitudinal force applied to the retrieval sleeve 58 will be converted by the braided fabrication to a radially contracting force. Hence, application of a longitudinal force to the retrieval sleeve 58 will radially contract the retrieval sleeve 58, and more importantly, radially contract the chassis 46 and blood filtering sleeve 54 to transition the bridge 18 from the expanded state to the collapsed state.

The retrieval sleeve 58 is secured to the chassis 46 at least at the upstream end 38 and extends past, or overhangs, the downstream end 42 of the chassis 46. For example the retrieval sleeve 58 can overhang the chassis 46 at the downstream end 42 of the bridge 18 by approximately 10-30 mm. In various implementations, the retrieval sleeve 58 can additionally be heat-set to match the profile, or shape, of the chassis 46 when in the expanded state.

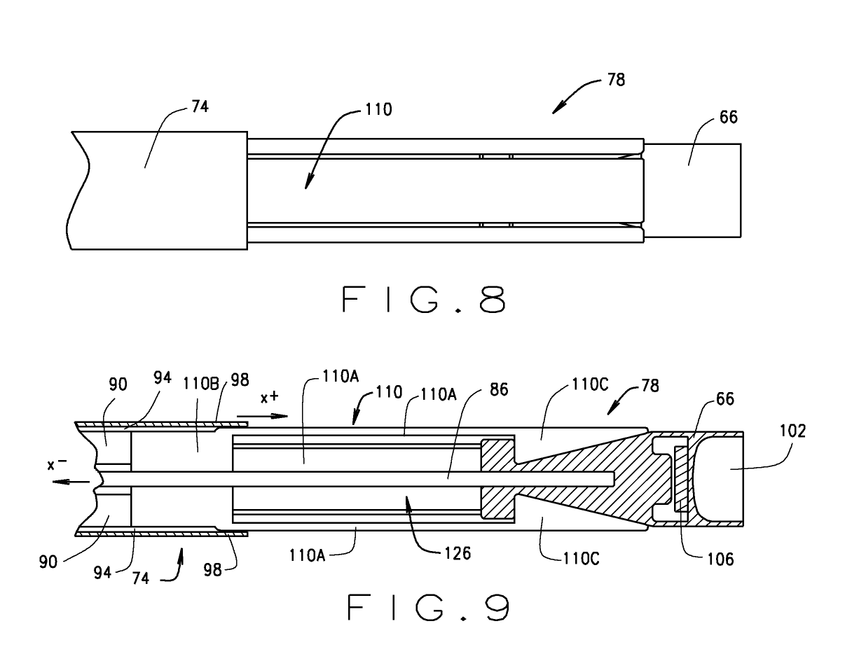

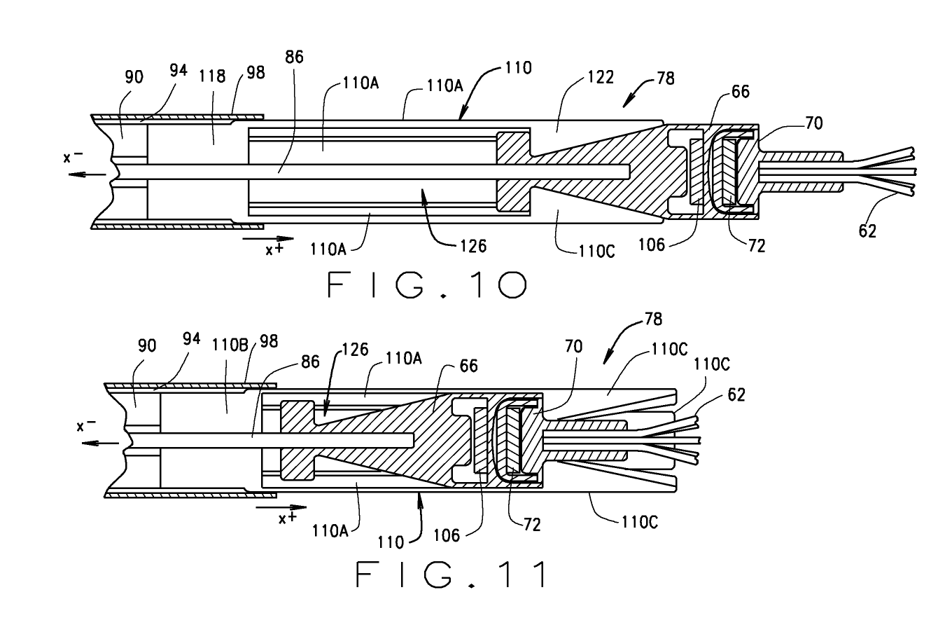

The retrieval sleeve 58 includes a plurality of retrieval strings 62 that are connected to or integrally formed with the retrieval sleeve 58 at their distal, or upstream, ends and joined together at their proximal, or downstream, ends. For example, in various embodiments, the retrieval strings 62 are woven into the braided mesh retrieval sleeve 58 at the upstream end 38 of the bridge 18. The retrieval strings 62 are joined at the proximal, or downstream, ends such that the retrieval strings 62 can be hooked, grasped, magnetically attached or otherwise connected to a bridge connector 66 (shown in FIG. 8) of the retrieval tool 22, as described further below. Once connected to the bridge connector 66, the retrieval tool 22 will apply a longitudinal force to the retrieval sleeve 58, whereby the braided mesh of the retrieval sleeve 58 will convert the longitudinal force into a radial force that assists in collapsing the bridge 18 to the cylindrical collapsed state such that the bridge 18 can be removed, or retrieved, from disposition within the aortic arch 26. In various embodiments, the proximal, or downstream, ends of the retrieval strings 62 are connected to, attached to, or joined together by, a magnetic button 70 that contains a small magnet 72, e.g., a small neodymium magnet disc (shown in FIG. 10). As described below, in such embodiments, the magnetic button 70 is magnetically connectable to the bridge connector 66 of the retrieval tool 22 such that the bridge 18 is easily connectable to the retrieval tool 22 for retrieval or removal of the bridge when desired.

Referring now to FIG. 7, as described above, the embolic protection system 10 further includes the bridge retrieval tool 22. It should be understood that although the bridge retrieval tool 22 is exemplarily described herein as part of the embolic protection system 10, and as being structured and operable to remove, or retrieve, the aortic arch bridge 18 from disposition within the aortic arch 26, the bridge retrieval tool 22 can be a stand-alone retrieval tool that is structured and operable to remove, or retrieve, other intra-luminal devices, or inter-tubular organ devices, e.g., other inter-arterial or inter-intestinal stents or devices, and remain within the scope of the present disclosure. More specifically, although the bridge retrieval tool 22 can be a stand-alone retrieval tool that is structured and operable to remove, or retrieve, other intra-luminal devices, or inter-tubular organ devices, e.g., other inter-arterial or inter-intestinal stents or devices, and remain within the scope of the present disclosure, for simplicity and clarity the retrieval tool 22 will be exemplarily described and illustrated herein as structured and operable to remove, or retrieve, the aortic arch bridge 18 described above.

In various embodiments, the retrieval tool 22 includes a multi-layer catheter, or tentacle, 74, a bridge coupling mechanism 78 disposed at a distal end of the multi-layer catheter 74, and a control handle 82 connected to a proximal end of the multi-layer catheter 74. The bridge coupling mechanism 78 is generally structured and operable to connect with the retrieval sleeve 58 of the blood filtering aortic arch bridge 18, described above, to retrieve the bridge 18 from disposition within the aortic arch 26. And, the control handle 82 is generally structured and operable to control the operation of the bridge coupling mechanism 78 and multi-layer catheter 74 to connect the bridge coupling mechanism 78 to the bridge 18, collapse the bridge 18 and retrieve or remove the bridge 18 from the aortic arch 26.

Referring now to FIGS. 9 and 13, in various embodiments, the multi-layer catheter 74 includes a flexible retention wire 86 slidably concentrically disposed within a flexible core 90 that is concentrically disposed within a flexible fixed tube 94. The flexible core 90 is fabricated of any flexible material suitable for slidably housing the retention wire 86 and providing support for the fixed tube 94 such that the retention wire 86 and the fixed tube 94 can flexibly bend but will not crimp or fold. The multi-layer catheter 74 additionally includes a movable flexible outer sheath 98 that is slidably concentrically disposed about the flexible fixed tube 94. The flexible core 90 is also provides support for the outer sheath 98 such that the outer sheath 98 can flexibly bend but will not crimp or fold.

Referring now to FIGS. 8 through 12, the bridge coupling mechanism 78 comprises the bridge connector 66, described above. The bridge connector 66 is affixed to a distal end of the retention wire 86 such that the bridge connector 66 extends from a distal end of the bridge coupling mechanism 78, which extends from a distal end of the catheter 74. As described above, the bridge connector 66 is structured and operable to hook, grasp, magnetically attach to or otherwise connect to the retrieval strings 62. Hence, the bridge connector 66 can be a hook, a clamp or clip, a magnetic, or any other device or mechanism suitable for securing the retrieval strings 62 of the bridge 18 to distal end of the retrieval tool catheter 74. Although the bridge connector 66 can be a hook, a clamp or clip, a magnetic, or any other device or mechanism suitable for securing the retrieval strings 62 of the bridge 18 to distal end of the retrieval tool catheter 74, the bridge connector 66 will be exemplarily described and illustrated herein a magnetic connector.

In such embodiments, the bridge connector 66 comprises a receptacle 102 and a magnet 106, e.g., a small neodymium magnet disc, disposed in or near a bottom of the receptacle 102. Therefore, the magnetic button 70 attached to the joined retrieval strings 62 of the aortic arch bridge 18, as exemplarily described above, is magnetically connectable to the bridge connector 66 of the retrieval tool 22. Particularly, when aortic arch bridge 18 is disposed within the aortic arch 26 and the retrieval tool catheter 74 is inserted into the aorta 30 such that the bridge connector 66 is in close proximity to the magnetic button 70, the magnetic button 70 is automatically magnetically drawn into the receptacle 102 via the attractive forces between the button magnet 72 and the connector magnet 106, as shown in FIG. 10. In various implementations, the shape of the button 70 and the shape of the receptacle 102 are complimentary such that the button 70 smoothly seats itself into the receptacle 102 without binding. For example, the button 70 can have a curved outer surface the seats smoothly into curved inner surface of the receptacle 102. Additionally, in various implementations, small radiopaque markers can be located at the contacting surfaces of the button 70 and the receptacle 102 that are used to verify, via fluoroscopy, a successful connection of the button 70 with the receptacle 102. Hence, the retrieval tool 22, particularly the catheter 74, is readily, automatically, and easily connected to the aortic arch bridge 18, particularly the retrieval strings 62 of the retrieval sleeve 58, when it is desired to remove, or retrieve, the bridge 18 from the aortic arch 26.

In various embodiments, the bridge coupling mechanism 78 includes a locking claw 110 affixed to a distal end of the fixed tube 94. The locking claw 110 is structured and operable to secure the connection of the bridge connector 66 with the retrieval strings 62 of retrieval sleeve 58. The locking claw 110 is formed to have a cylindrical shape wherein an outside diameter of the locking claw 110 is smaller than in inside diameter of the outer sheath 98 such that the outer sheath 98 can be extended over the locking claw 110, as described further below. In various embodiments, the locking claw 110 comprises a plurality of fingers 110A that extend from a base 110B and have wedge-shaped retaining teeth 110C formed at distal ends.

The locking claw 110 is structured such that the fingers 110A are biased to a normal position, wherein the locking claw 110 has the cylindrical shape, as shown in FIGS. 8 through 12. However, due to the wedge shape of the retaining teeth 110C, the fingers 110A can temporarily spread apart by pulling the bridge connector 66 into an interior chamber 126 of the locking claw 110, whereafter the biasing of the fingers 110A will return the fingers 110A to the normal position. More specifically, once the bridge connector 66 has secured the retrieval strings 62 of the bridge 18, as described above, the retention wire 86 is pulled in the X⁻ direction, via operation of the control handle 82, as described below. This will consequently withdraw the bridge connector 66 and the attached retrieval strings 62 in the X⁻ direction forcing the fingers 110A to spread until the bridge connector 66 and the attached retrieval strings 62 are disposed within the interior chamber 126, whereafter the fingers 110A will return to their normal position. Once the bridge connector 66 and the attached retrieval strings 62 have been withdrawn into the interior chamber 126 and the fingers 110A have returned to the normal position, the wedge shape of the retaining teeth 110C will prevent that bridge connector 66 and the attached retrieval strings 62 from being pulled back out of the interior chamber 126. Accordingly, the aortic arch bridge 18 will be fixedly connected to the retrieval tool catheter 74.

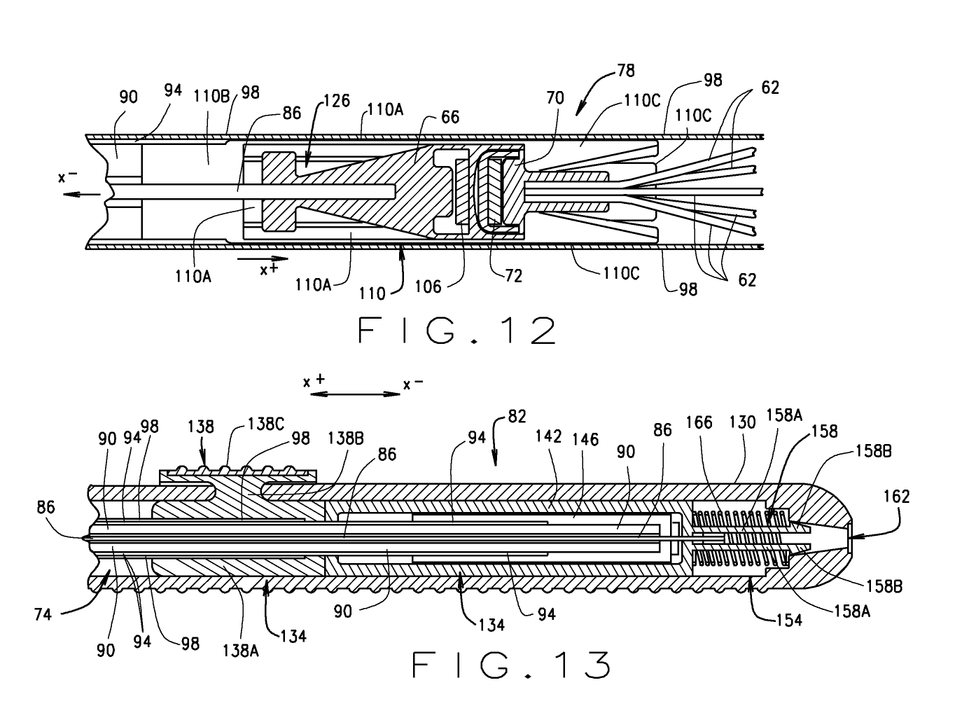

For example, in the embodiments wherein the aortic arch bridge 18 includes the magnetic button 70 and the bridge connector 66 includes the magnet 106, once the magnetic button 70 is magnetically connected to the magnetic bridge connector 66 and seated within the receptacle 102, the retention wire 86 is pulled in the X⁻ direction, via operation of the control handle 82. This will consequently pull the magnetically connected button 70 and bridge connector 66 in the X⁻ direction forcing the fingers 110A to spread. Continued pulling of the retention wire 86 in the X⁻ direction will withdraw the magnetically connected button 70 and bridge connector 66 into the interior chamber 126, as illustrated in FIGS. 11 and 12. Subsequently, the biased fingers 110A will return to their normal position, whereby the wedge-shaped retaining teeth 110C will prevent the magnetically connected button 70, having the retrieval strings 62 of the bridge retrieval sleeve 58 attached thereto, from being pulled back out of the interior chamber 126. Accordingly, the aortic arch bridge 18 will be fixedly connected to the retrieval tool catheter 74.

Once the bridge connector 66 and the attached retrieval strings 62 have been pulled into the interior chamber 126 such that the aortic arch bridge 18 is fixedly connected to the retrieval tool catheter 74, the outer sheath 98 can be advanced, via operation of the control handle 82, in the X⁺ direction over the locking claw 110, as shown in FIG. 12. Furthermore, the outer sheath 98 can be further advanced in the X⁺ direction over the aortic arch bridge 18, as described further below. Importantly, as the outer sheath 98 is advanced in the X⁺ direction, due to the retention of the retrieval strings 62 by the coupling mechanism 78, a longitudinal force will be applied by the outer sheath 98 to the retrieval strings 62, and more importantly to the retrieval sleeve 58. Consequently, as described above, the braided mesh of the retrieval sleeve 58 will convert the longitudinal force into a radial force that collapses the bridge 18 to the cylindrical collapsed state such that outer sheath 98 can be advanced over the bridge 18, whereafter the bridge 18 can be removed, or retrieved, from disposition within the aortic arch 26. It is envisioned that in various embodiments, to aid in the advancement of the outer sheath 98 over the bridge 18, the distal end of outer sheath 98 can include a plurality slits that allow the distal end to slight expand or widen as the outer sheath 98 contacts the retrieval strings and sleeve 62 and 58, thereby assisting in smooth advancement of the outer sheath 98 over the bridge 18.

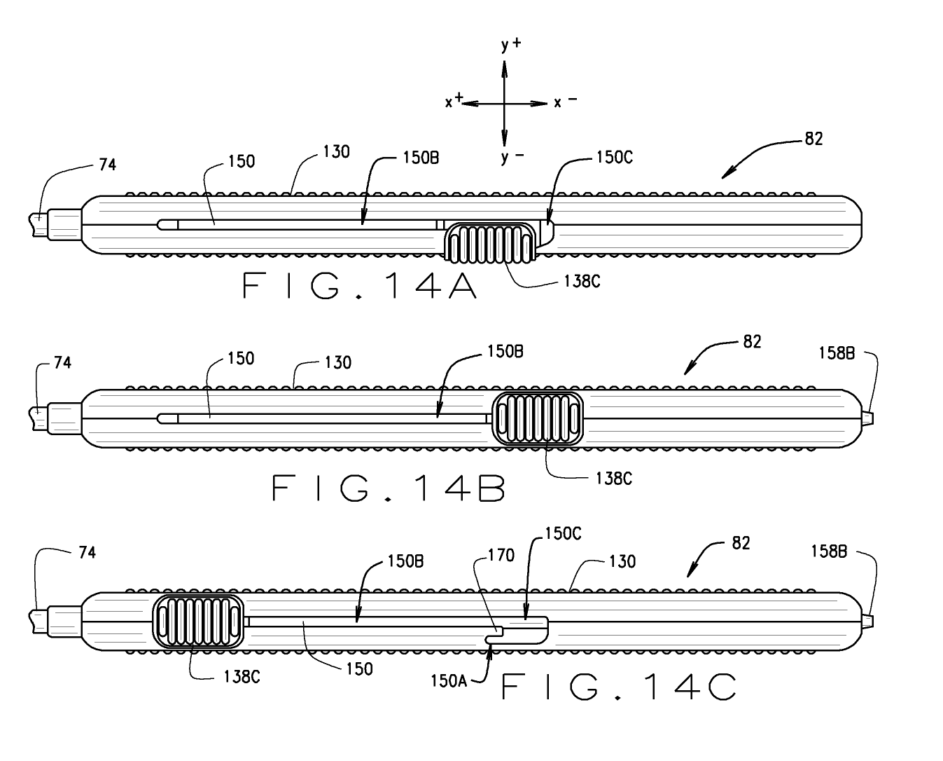

Referring now to FIGS. 13 through 14C, the control handle 82 generally includes a housing 130 and catheter control module 134 slideably disposed within the housing 130. The control module 134 is structured and operable by an operator, e.g., a physician, surgeon or other medical personnel, to control movement of the retention wire 86 in the X⁻ direction and the movement of the outer sheath 98 in the X⁺ direction to connect the catheter 74 to the aortic arch bridge 18 and collapse the bridge 18 for removal, as described above. It should be noted that the control module 134 is further structured and operable by the operator to control movement of the retention wire 86 in the X⁺ direction and the movement of the outer sheath 98 in the X⁻ direction.

In various embodiments, the control module 134 includes a thumb controller 138, a retention wire fixture 142 and a core and fixed tube fixture 146. The thumb controller 138 includes an outer sheath fixture 138A that is structured and operable to fixedly retain a proximal end of the outer sheath 98 such that the outer sheath 98 will be advance and retracted in the X⁺ and X⁻ directions as the thumb controller 138 is moved in the X⁺ and X⁻ directions. The thumb controller additionally includes a neck 138B extending from the outer sheath fixture 138A through a J-shaped guide slot 150 in the housing 130. The thumb controller 138 further includes a thumb pad 138C connected to a distal end of the neck 138B such that the thumb pad 138C is disposed on the exterior of the housing and is accessible to the operator holding the control handle 82. The thumb controller 138 is slideably disposed within an interior cavity 154 of the housing 130. Particularly, via manipulation of the thumb pad 138C by the operator, the thumb controller 138 can be moved in the X⁺ direction and the X⁻ direction. More specifically, the operator can move the thumb pad 138C in the X⁺ and X⁻ directions causing the neck 138B to correspondingly slide within the J-shaped guide slot 150 in the X⁺ and X⁻ directions, which in turn causes the outer sheath fixture 138A, and importantly the outer sheath 98, to correspondingly move in the X+ and X⁻ directions. The J-shaped guide slot 150 is structured and operable to guide the movement of the thumb controller 138.

The retention wire fixture 142 is slideably disposed within the interior cavity 154 of the housing 130 and is structured and operable to fixedly retain a proximal end of the retention wire 86 such that the retention wire 86 will be moved in the X⁺ and X⁻ directions as the retention wire fixture 142 is moved in the X⁺ and X⁻ directions, as described below. In various embodiments, the retention wire fixture 142 includes a snap-lock tail 158 that is structured and operable to selectably retain, or lock, the retention wire 86 and the retention wire fixture 142 in a ‘Withdrawn’ position, wherein the bridge connector 66 connected to the distal end of the retention wire 86 and retrieval strings 62 attached to the bridge connector 66 are withdrawn into the interior chamber 126 of the locking claw 110, as described above. In various embodiments, the snap-lock tail 158 comprises a pair of opposing tines 158A that extend from a base 142A of the retention wire fixture 142 and have wedge-shaped locking teeth 158B formed at distal ends. As illustrated in FIG. 13, when the thumb controller 138 and the retention wire fixture are in a ‘Home’ position, the locking teeth 158B protrude into, but do not extend through, a lock orifice 162 formed in a rear end of the housing 130.

As described further below, to lock the retention wire and fixture 86 and 142 in the Withdrawn position, the operator moves the thumb controller 138 in the X⁻ direction such that the outer sheath fixture 138A pushes the retention wire fixture 142 in the X⁻ direction. As the retention wire fixture 142 moves in the X⁻ direction, the tines and locking teeth 158A and 158B of the snap-lock tail 158 are pushed through the lock orifice 162. Subsequently, the locking teeth 158B will extend out of the lock orifice 162, whereafter the resiliency of the tines 158A will push the locking teeth 158B radially outward such that the wedge shape of the locking teeth 158B engage the rear end of the housing 130, as shown in FIGS. 14B and 14C, thereby locking the retention wire and fixture 86 and 142 in the Withdrawn position.

In various embodiments, the control handle 82 includes a biasing spring 166 disposed around the snap-lock tail 158 within the interior cavity 154 of the housing 130 such that the biasing spring 166 will bias the retention wire fixture 142 to the Home position. Moreover, the biasing spring 166 is structured and operable to maintain the retention wire fixture 142 in the Home position until the operator selectively moves the retention wire fixture 142 to the Withdrawn position, as described above.

The core and fixed tube fixture 146 is fixedly disposed within the housing interior cavity 154 such that it is not movable in the X⁺ and X⁻ directions. Particularly, the core and fixed tube fixture 146 is structured and operable to fixedly retain the flexible fixed tube 94 and the flexible core 90 of the multi-layer catheter 74 such that the flexible fixed tube and core 94 and 90 cannot move in the X⁺ and X⁻ directions. Moreover, the core and fixed tube fixture 146 is structured and operable to maintain the flexible fixed tube and core 94 and 90 stationary as the outer sheath 98 and the retention wire 86 are controllably moved over and within the fixed tube 94 and core 90 in the X⁺ and/or X⁻ directions, via operator manipulation of the thumb pad 138C as described above.

With further reference FIGS. 13 and 14A through 14C, the J-shaped guide slot 150 comprises a short Home channel 150A that is connected to a long sheath extension channel 150B via a switching channel 150C. FIG. 14A illustrates the thumb pad 138C in the Home position wherein the neck 138B is positioned within the Home channel 150A and is separated from the sheath extension channel 150B by interstitial tab 170 formed in the housing 130 between the Home channel 150A and the sheath extension channel 150B. Particularly, when the thumb pad 138C is in the Home position, the outer sheath fixture 138A is also in the Home position within the interior cavity 154 of the housing 130, as shown in FIG. 13.

FIG. 14B shows the thumb pad 138C, and consequently, the outer sheath fixture 138A moved from the Home position to the Withdrawn position. As shown in FIG. 13, when the thumb controller 138 is in the Home position, the outer sheath fixture 138A is in contact with the retention wire fixture 142. Hence, when the thumb controller 138 is moved from the Home position in the X⁻ direction, the neck 138B is moved along the Home channel 150A and the retention wire fixture 142 is pushed by the outer sheath fixture 138A in the X⁻ direction, thereby moving the retention wire 86 in the X⁻ direction. As described above, movement of the retention wire 86 in the X⁻ direction withdraws the connected retrieval strings 62 and bridge connector 66, e.g., the magnetically connected magnetic bridge connector 66 and magnetic button 70, into the interior chamber 126 of the bridge coupling mechanism locking claw 110, as illustrated in FIG. 11. Additionally, as movement of thumb controller 138 in the X⁻ direction pushes the retention wire fixture 142 in the X⁻ direction, the snap-lock tail 158 is pushed through the lock orifice 162 in the rear end of the control handle housing 130 until the retention wire fixture 142 and retention wire 86 are locked in the Withdrawn position, as illustrated in FIGS. 14B and 14C.

Once the retention wire fixture 142 and retention wire 86 have been moved to the Withdrawn position, whereby the connected bridge connector 66 and retrieval strings have been withdraw into, and secured within, the interior chamber 126 of the locking claw 110, the thumb pad 138C can be moved in the Y⁺ direction to move the neck 138B of the thumb controller 138 from the Home channel 150A to the sheath extension channel 150B, via the switching channel 150C. Subsequently, the thumb pad 138C can be pushed in the X⁺ direction by the operator, thereby moving the outer sheath fixture 138A in the X⁺ direction and advancing the outer sheath 98 along the stationary fixed tube 94 and core 90 in the X⁺ direction, as illustrated in FIG. 12. As described above, movement of the outer sheath in the X⁺ direction with the retrieval strings 62 securely retained by the bridge coupling mechanism 78 will exert a longitudinal force on the braided bridge retrieval sleeve 58. As further described above, the braided mesh of the retrieval sleeve 58 will convert the longitudinal force to a radially contracting force such that the bridge 18 is progressively collapsed from the expanded state to the collapsed state as the outer sheath 98 is advanced in the X⁺ direction such that the outer sheath 98 can be over the collapsed bridge 18 until the entire bridge 18 is disposed and retained within the interior lumen of the outer sheath 98. Thereafter, the multi-layer catheter 74, having the collapsed bridge 18 retained within the outer sheath 98, can be withdrawn from the aorta 30, thereby removing, or retrieving, the bridge 18 from the aortic arch 26.

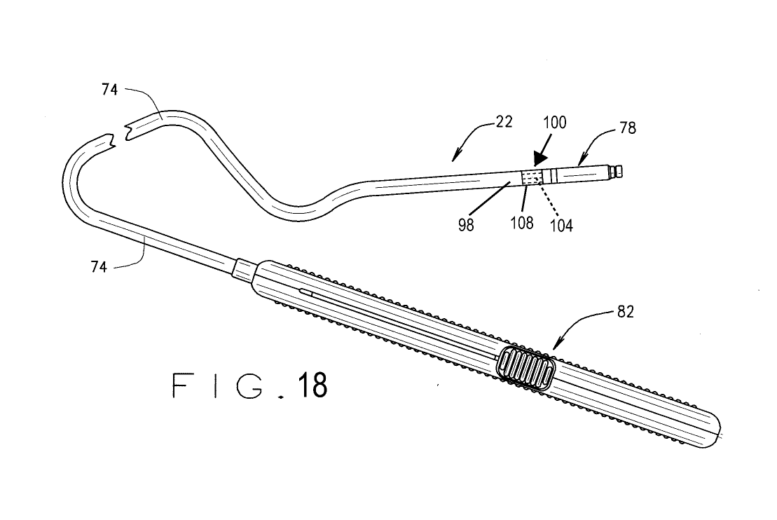

Referring now to FIG. 18, in various embodiments, the distal end of the outer sheath 98 can be structured and operable to expand and contract, or open and close, to and from a sub conical shape to aid in the retrieval of the bridge 18. For example, in various embodiments, the outer sheath 98 can include an expandable mouth 100 formed, or disposed, at the distal end. In such embodiments, the mouth 100 is structured and operable such that when the outer sheath 98 is moved in the X⁺ direction to advance the outer sheath 98 over the collapsing bridge 18, as described above, the mouth 100 will open, i.e., the distal end of the outer sheath 98 will expand, to aid in the advancement of the outer sheath 98 over the collapsing bridge 18. Additionally, the mouth 100 is structured and operable to remain, or be maintained, in a closed state, where the outside diameter of the mouth 100 is substantially the same as the outside diameter of the remainder of the outer sheath 98, prior to the outer sheath 98 being advanced over the bridge 18 and after the outer sheath 98 has been advanced over the entire bridge 18. Hence, in such embodiments, a larger insertion port incision in the patient will not be needed to accommodate the multi-layer catheter 74.

As described above, the mouth 100 is structured and operable to aid in the retrieval of the bridge 18. That is, it will selectively configure the distal end of the outer sheath 98 to more easily accommodate the retrieval strings and sleeve 62 and 58 such that the outer sheath 98 can be easily advanced over the collapsing bridge 18 without catching or snagging on the retrieval strings or sleeve 62 and 58.

In various implementations, the mouth 100 can comprise a plurality of radially equidistant longitudinal slits 104 cut down the distal end, or tip, of the outer sheath 98 that are structured and operable to allow the mouth 100 open and close as desired. In various implementations, the mouth 100 can additionally comprise a thin elastomer, e.g., silicone, membrane or sleeve 108 disposed over the slits 104 to retain the mouth in the closed state prior to advancement of the outer sheath 98 over the bridge 18 and return the mouth 100 to the closed state once the outer sheath 98 has been advanced entirely over the bridge 18. In various other implementations, to further aid in the ease of advancing the outer sheath 98 over the bridge 18, the mouth 100 can further comprise a flexible, smooth, and expandable woven mesh (not shown) that lines the interior of the of the mouth 100, i.e., the interior of the distal end of the outer sheath 98 where the mouth 100 is disposed. Disposition of the mesh on the interior of the mouth 100 will further reduce catching or snagging of the outer sheath 98 on the retrieval strings or sleeve 62 and 58 as the outer sheath 98 is advanced over the bridge 18.

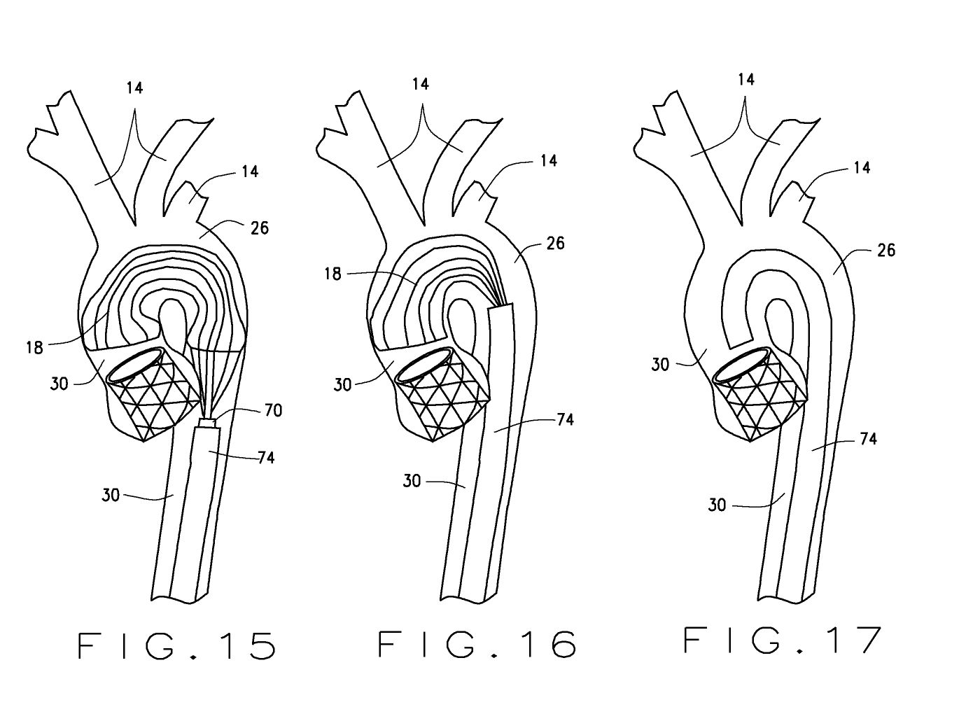

In operation, to remove, or retrieve, the aortic arch bridge 18 from within the aortic arch 26, the multi-layer catheter 74 is inserted into the aorta 30 via known procedures for inserting known catheters into the aorta, e.g., through an iliac artery via an incision near the patient's groin. Subsequently, the operator positions the multi-layer catheter 74 to connect the bridge connector 66 with the retrieval strings 62 of the bridge 18, as shown in FIG. 15. For example, in the embodiments wherein bridge connector 66 comprises the magnet 106 and the retrieval strings 62 are joined together by the magnetic button 70, the magnetic bridge connector 66 is placed in close proximity to the magnetic button 70, whereby the attractive magnetic forces between the magnets 72 and 106 automatically connect the retrieval strings 62 of the bridge 18 to the bridge connector coupling device 78.

Next, the operator moves the thumb controller 138 from the Home position to the Withdrawn position, via the thumb pad 138A, thereby moving the retention wire fixture 142 in the X⁻ direction. As described above movement of the retention wire fixture 142 in in the X⁻ direction, moves the retention wire 86 within the flexible core 90 in the X⁻ direction. This, in turn, withdraws the connected bridge connector 66 and retrieval strings 62, e.g., the magnetically connected bridge connector 66 and magnetic button 70, into the interior chamber 126 of the locking claw 110, whereby the retrieval strings 62 are securely connected to the catheter 74. Once the retrieval strings 62 have been secured within the locking claw 110, the operator moves the neck 138B of the thumb controller 138 from the Home channel 150A, through the switching channel 150C, into the extension channel 150B, via the thumb pad 138C. Next, the operator slowly pushes the thumb pad 138 and outer sheath fixture 138A in the X⁺ direction along the extension channel 150B, thereby slowly advancing the outer sheath 98 over the retrieving strings 62 and the portion of the retrieval sleeve 58 that overhangs the downstream end of the bridge chassis 46.

As described above, advancement of the outer sheath 98 over the retrieving strings 62 and the overhanging portion of the retrieval sleeve 58 causes the braided retrieval sleeve to exert radially collapsing forces on the chassis 46 and blood filtering sleeve 54. Consequently, the radially collapsing forces progressively collapse the bridge 18 to the collapsed state as the outer sheath 98 is advanced over the progressively collapsing bridge 18, as shown in FIG. 16. Finally, via further movement of the thumb pad 138C in the X⁺ direction along the extension channel 150B, the outer sheath 98 is advanced over the entire collapsed bridge 18, as shown in FIG. 17. Thereafter, the multi-layer catheter 74 and the collapsed bridge 18 retained within the outer sheath 98 of the catheter 74 are removed from the patient.

Alternatively, to remove, or retrieve, other intra-luminal devices from within the respective tubular organ, the multi-layer catheter 74 is inserted into respective tubular organ via known procedures for inserting known catheters. Subsequently, the operator positions the multi-layer catheter 74 to connect the bridge connector 66 with retrieval strings of the respective intra-luminal device, whereafter the operator connects the bridge connector 66 with the retrieval strings of the respective intra-luminal device.

Next, the operator moves the thumb controller 138 from the Home position to the Withdrawn position, via the thumb pad 138A, thereby moving the retention wire fixture 142 in the X⁻ direction. As described above movement of the retention wire fixture 142 in in the X⁻ direction, moves the retention wire 86 within the flexible core 90 in the X⁻ direction. This, in turn, withdraws the connected bridge connector 66 and retrieval strings into the interior chamber 126 of the locking claw 110, whereby the retrieval strings are securely connected to the catheter 74. Once the retrieval strings have been secured within the locking claw 110, the operator moves the neck 138B of the thumb controller 138 from the Home channel 150A, through the switching channel 150C, into the extension channel 150B, via the thumb pad 138C. Next, the operator slowly pushes the thumb pad 138C and outer sheath fixture 138A in the X⁺ direction along the extension channel 150B, thereby slowly advancing the outer sheath 98 over the retrieving strings and the respective intra-luminal device until the entire intra-luminal device is disposed within the outer sheath 98. Thereafter, the multi-layer catheter 74 and the respective intra-luminal device retained within the outer sheath 98 are removed from the patient.

It is envisioned that in various embodiments, the control handle 82 can further comprise a retention wire withdrawal device (e.g., a slider, lever, wheel, etc.) structured and operable to continuously move the retention wire 86 in the X⁻ direction as the outer sheath 98 is advanced in the X⁺ direction. Hence, in such embodiments, as the outer sheath 98 is being advance in the X⁺ direction by movement of the thumb pad 138C along the extension channel 150B, the retention wire withdrawal device simultaneously moves the retention wire 86 in the X⁻ at substantially the same rate of movement. Accordingly, in such embodiments, the natural elongation of the bridge 18 as the bridge 18 collapses is compensated for by withdrawal of the retention wire 86, and more importantly withdrawal of the bridge connector 66 and retrieval strings 62, into the outer sheath 98. That is, as the outer sheath 98 is advanced in the X⁺ direction over the bridge 18, the elongation of collapsing bridge 18 is compensated for by withdrawing the downstream end 42 of the bridge in the X⁻ direction into the outer sheath 98 at the same rate as the outer sheath 98 is advanced in the X⁺ direction. It is envisioned that the control handle 82 can comprise and combination of gears, levers, pulleys, etc that are cooperatively operable to ensure an optimum ratio between the advancement of the outer sheath 98 in the X⁺ direction and the opposing withdrawal of bridge downstream end 42 in the X⁻ direction. Alternatively, it is envisioned that the elongation of the collapsing bridge 18 can be compensated for by the operator pulling the entire control handle 82 and attached multi-layer catheter 74 in the X⁻ direction as the outer sheath 98 is advanced in the X⁺ direction.

The description herein is merely exemplary in nature and, thus, variations that do not depart from the gist of that which is described are intended to be within the scope of the teachings. Such variations are not to be regarded as a departure from the spirit and scope of the teachings.

1. An embolic protection system, said system comprising
a collapsible blood filtering aortic arch bridge structured and operable to bend to comply with the curvature of an aortic arch of a patient into which the bridge is disposable, the bridge comprising:
a chassis that is expandable and collapsible, the chassis structured to provide the bridge with a dumbbell-like shape when expanded having a tubular waist, a first conical end formed at a first end of the waist, and a second conical end formed at an opposing second end of the waist such that only a periphery of the first and second ends contact the intima of the aortic arch when the bridge is disposed and expanded within the aortic arch;
a blood filtering sleeve attached to the chassis, the blood filtering sleeve structured and operable to filter blood flowing through the bridge into aortic arch vessels of the patient when the bridge is disposed within the aortic arch; and
a retrieval sleeve disposed over an exterior of the chassis, the retrieval sleeve structured and operable to collapse the bridge to a cylindrical form for retrieval of the bridge from the aortic arch; and
a retrieval tool structured and operable to retrieve the bridge from disposition within the aortic arch, the retrieval tool comprising:
a multi-layer catheter;
a bridge coupling mechanism disposed at an end of the multi-layer catheter, the bridge coupling mechanism structured and operable to connect with the retrieval sleeve to retrieve the bridge from disposition within the aortic arch; and
a control handle connected to the catheter and structured and operable to control longitudinal movement of the retention wire and the outer sheath.

2. The system of claim 1, wherein the chassis is fabricated of shape memory material shape-set to provide a shape memory cage having the dumbbell-like shape that is transformable between the dumbbell-like shape and the cylindrical form.

3. The system of claim 1, wherein the blood filtering sleeve is fabricated of a biocompatible mesh that is a knitted mesh such that the blood filtering sleeve will elastically expand and contract longitudinally and not laterally when the bridge is collapsed from the to the cylindrical form for retrieval of the bridge.

4. The system of claim 1, wherein the retrieval sleeve is fabricated of a biocompatible mesh that is braided such that longitudinal force applied to an end of the sleeve will be converted to radial force utilized to collapse the bridge to the cylindrical form for retrieval of the bridge from disposition within the aortic arch.

5. The system of claim 4, wherein the retrieval sleeve comprises a plurality of retrieval strings connected to the retrieval sleeve and joined together at proximal ends, the retrieval strings structured and operable to connect with the bridge connector of the retrieval tool and transfer longitudinal force applied by the retrieval tool, via connect of the retrieval strings with the bridge connector, to the retrieval sleeve to collapse the bridge to the cylindrical form for retrieval of the bridge from disposition within the aortic arch.

6. The system of claim 5, wherein retrieval sleeve further comprises a magnetic button connected to the joined proximal ends of the retrieval strings, and the bridge connector of the retrieval tool comprises a magnetic receptacle magnetically connectable to the magnetic button to connect the retrieval tool with the retrieval sleeve for retrieval of the bridge from disposition within the aortic arch.

7. The system of claim 1, wherein the multi-layer catheter comprises:
a retention wire;
a flexible core concentrically disposed around the retention wire,
a flexible fixed tube concentrically disposed around the flexible core: and
a movable flexible outer sheath concentrically disposed around the flexible fixed tube.

8. The system of claim 7, wherein the bridge coupling mechanism comprises a bridge connector affixed to distal end of the retention wire, the bridge connector structured and operable to connect the catheter to the bridge.

9. The system of claim 8, wherein the bridge coupling mechanism further comprises a locking claw affixed to a distal end of the fixed tube and structured and operable to secure the connection of the bridge connector with the retrieval sleeve to retrieve the bridge from disposition within the aortic arch.

10. The system of claim 9, wherein the control handle comprises:
a housing and a catheter control module slideably disposed within the housing, the control module structured and operable to control the longitudinal movement of the retention wire and the outer sheath; and
a retention wire fixture structured and operable to retain the retention wire in a locked position wherein the locking claw secures the connection of the bridge connector with the retrieval sleeve.

11. A collapsible blood filtering aortic arch bridge, said bridge comprising:
an expandable and collapsible chassis structured to provide the bridge with a dumbbell-like shape when expanded having a tubular waist, a first conical end formed at a first end of the waist, and a second conical end formed at an opposing second end of the waist such that only a periphery of the first and second ends contact the intima of an aortic arch when the bridge is disposed and expanded within the aortic arch of a patient, the chassis structured and operable to bend to comply with the curvature of the aortic arch of the patient;
a blood filtering sleeve disposed over one of an interior or an exterior of the chassis, the blood filtering sleeve structured and operable to filter blood flowing through the bridge into aortic arch vessels of the patient when the bridge is disposed within the aortic arch; and
a retrieval sleeve disposed over the exterior of the chassis, the retrieval sleeve structured and operable to collapse the bridge to a cylindrical form for retrieval of the bridge from the aortic arch.

12. The bridge of claim 11, wherein the chassis is fabricated of shape memory material shape-set to provide a shape memory cage having the dumbbell-like shape that is transformable between the dumbbell-like shape and the cylindrical form.

13. The bridge of claim 11, wherein the blood filtering sleeve is fabricated of a biocompatible mesh that is a knitted mesh such that the blood filtering sleeve will elastically expand and contract longitudinally and not laterally when the bridge is collapsed from the to the cylindrical form for retrieval of the bridge.

14. The bridge of claim 11, wherein the retrieval sleeve is fabricated of a biocompatible mesh that is braided such that longitudinal force applied to an end of the sleeve will be converted to radial force utilized to collapse the bridge to the cylindrical form for retrieval of the bridge from disposition within the aortic arch.

15. The bridge of claim 14, wherein the retrieval sleeve comprises a plurality of retrieval strings connected to the retrieval sleeve and joined together at proximal ends, the retrieval strings structured and operable to connect with a bridge retrieval tool and transfer longitudinal force applied by the retrieval tool to the retrieval sleeve to collapse the bridge to the cylindrical form for retrieval of the bridge from disposition within the aortic arch.

16. The bridge of claim 15, wherein retrieval sleeve further comprises a magnetic button connected to the joined proximal ends of the retrieval strings for magnetic connection to the retrieval tool for retrieval of the bridge from disposition within the aortic arch.

17. The bridge of claim 11, wherein the first conical end of the chassis has a larger outside diameter than the second conical end such that the bridge conforms to the physical shape and structure of the aortic arch.

18. A collapsible blood filtering aortic arch bridge, said bridge comprising:
an expandable and collapsible chassis fabricated of shape memory material and structured to provide the bridge with a dumbbell-like shape when expanded having a tubular waist, a first conical end formed at a first end of the waist, and a second conical end formed at an opposing second end of the waist such that only a periphery of the first and second ends contact the intima of an aortic arch when the bridge is disposed and expanded within the aortic arch of a patient, the first conical having a larger outside diameter than the second conical end such that the bridge conforms to the physical shape and structure of the aortic arch, and the waist being flexible so that the bridge can bend to comply with the curvature of the aortic arch of the patient;
a blood filtering sleeve disposed over one of an interior or an exterior of the chassis, the blood filtering sleeve structured and operable to filter blood flowing through the bridge into aortic arch vessels of the patient when the bridge is disposed within the aortic arch; and
a retrieval sleeve disposed over the exterior of the chassis, the retrieval sleeve structured and operable to collapse the bridge to a cylindrical form for retrieval of the bridge from the aortic arch, the retrieval sleeve comprises a plurality of retrieval strings connected to the retrieval sleeve and joined together at proximal ends, the retrieval strings structured and operable to connect with a bridge retrieval tool and transfer longitudinal force applied by the retrieval tool to the retrieval sleeve to collapse the bridge to the cylindrical form for retrieval of the bridge from disposition within the aortic arch.

19. The bridge of claim 18, wherein the blood filtering sleeve is fabricated of a biocompatible mesh that is a knitted mesh such that the blood filtering sleeve will elastically expand and contract longitudinally and not laterally when the bridge is collapsed from the to the cylindrical form for retrieval of the bridge.

20. The bridge of claim 18, wherein the retrieval sleeve is fabricated of a biocompatible mesh that is braided such that longitudinal force applied to an end of the sleeve will be converted to radial force utilized to collapse the bridge to the cylindrical form for retrieval of the bridge from disposition within the aortic arch.

21. The bridge of claim 18, wherein retrieval sleeve further comprises a magnetic button connected to the joined proximal ends of the retrieval strings for magnetic connection to the retrieval tool for retrieval of the bridge from disposition within the aortic arch.

22. A retrieval tool structured and operable to retrieve an inner tubular organ (ITO) device disposed within an interior of a tubular organ of a patient, said tool comprising:
a multi-layer catheter comprising:
a flexible retention wire;
a flexible core concentrically disposed around the retention wire,
a flexible fixed tube concentrically disposed around the flexible core; and
a flexible movable outer sheath concentrically disposed around the fixed tube;
an ITO device coupling mechanism disposed at a distal end of the multi-layer catheter, the ITO device coupling mechanism structured and operable to connect with the ITO device to retrieve the ITO device from disposition within the interior of a tubular organ of the patient; and
a control handle connected to the catheter and structured and operable to control longitudinal movement of the retention wire and the outer sheath.

23. The system of claim 22, wherein the ITO device coupling mechanism comprises a bridge connector affixed to distal end of the retention wire, the bridge connector structured and operable to connect the catheter to the bridge.

24. The system of claim 23, wherein the ITO device coupling mechanism comprises a locking claw affixed to a distal end of the fixed tube and structured and operable to secure the connection of the bridge connector with the retrieval sleeve to retrieve the bridge from disposition within the aortic arch.

25. The system of claim 24, wherein the control handle comprises a housing and a catheter control module slideably disposed within the housing, the control module structured and operable to control the longitudinal movement of the retention wire and the outer sheath.

26. The system of claim 25, wherein the control handle comprises a retention wire fixture structured and operable to retain the retention wire in a locked position wherein the locking claw secures the connection of the bridge connector with the retrieval sleeve.
